(19) United States
(12) Patent Application Publication
(10) Pub. No.: US 2020/0008967 A1
(43) Pub. Date: Jan. 9, 2020

(54) THERAPEUTIC SPLINT

(71) Applicant: CENTAUR CLINICAL CRO, Gardanne (FR)

(72) Inventor: Ouadah HADJEBI, Gardanne (FR)

(21) Appl. No.: 16/483,522

(22) PCT Filed: Feb. 6, 2018
(86) PCT No.: PCT/EP2018/052891

§ 371 Date: Aug. 5, 2019

(30) Foreign Application Priority Data
Feb. 6, 2017 (FR) 1750983

(51) Int. Cl. A61F 5/058; A61F 7/02

(52) U.S. Cl. CPC A61F 5/058; A61F 5/0585; A61F 5/05866; A61F 7/02; A61F 2007/0036; A61F 2007/0285; A61F 2007/0268; A61F 2007/0298; A61F 2007/0225

(57) ABSTRACT

A therapeutic splint for the treatment of one or more joint areas of a patient. The splint includes longitudinal housings extending along a longitudinal axis (L) and arranged parallel to this longitudinal axis (L); one or more modular cartridges with therapeutic effect suitable for being arranged in the housings according to the joint area (s) to be treated. The splint further includes at least one transverse and/or oblique housing extending along one or more transverse and/or oblique axes (O), respectively, with respect to the longitudinal axis (L) of the splint. The transverse and/or oblique housing is arranged so as to cover an area extending around a joint to be treated. The modular cartridge or cartridges are arranged in the transverse and/or oblique housings according to the joint area(s) to be treated.

### TECHNICAL FIELD OF THE INVENTION

The present invention relates to the field of therapeutic devices capable of relieving people suffering from neuralgia, fractured limbs or injuries, for example resulting from sports activities. More particularly, the present invention relates to a therapeutic splint for the treatment of one or more anatomical areas.

### BACKGROUND OF THE INVENTION

A splint is a therapeutic device designed to immobilize or stabilize a painful, unstable or healing joint or anatomical area. Splints are therefore generally used to prevent or in the event of traumatic injuries (such as impacts, blows, falls, stumbles, etc.), sprains, tendonitis, inflammation or fracture of an anatomical limb of a human body. These splints come in different sizes and shapes depending on the limb to be immobilized (arm, hand, leg, etc.) and the user's morphology. These splints are generally envelopes that sometimes include pockets in which therapeutic gels are arranged, for example to cool, warm or treat a local anatomical surface of the human body.

Despite the many advances that have been made in this medical field, there are gaps in the use of therapeutic splints to relieve and/or heal physical pain. Indeed, some splints are difficult to place and do not allow for optimal treatment of all types of anatomical areas or surfaces. In addition, the splints of the prior art have the disadvantage of being bulky and cumbersome, which makes their use impractical and uncomfortable for the patient.

The prior art includes therapeutic devices, for example as described in patent application US 2010/0152823, especially in cryotherapy, to reduce swelling or inflammation caused by injury or surgery. This device includes a therapeutic envelope that is attached around the human body (in particular at the hip), for example by straps with fastening mechanisms, such as a hook and a loop of Velcro® fabric, a zipper, laces, etc. This device further includes at least one longitudinal chamber extended along a longitudinal axis of the device, which can contain at least one cold or hot pack and which can be subdivided into several chambers and closed by detachable fastening mechanisms. However, this therapeutic device has the disadvantage of being suitable only for treating anatomical surfaces along a longitudinal axis of a limb of the human body. It cannot therefore be used to relieve any type of limb or anatomical area. On the other hand, this device only treats a localized area of the body. To treat larger areas of the body, the dimensions of the device must be completely changed. As a result, this device is not adjustable and is not compatible with all types and sizes of anatomical surfaces or areas of the human body.

The prior art also includes a therapeutic device including one or more envelopes containing a reusable hot or cold thermal pack for the thermal treatment of inflamed or painful muscles. The envelope includes closure means to lock the thermal pack and attachment means to allow the envelope to attach directly to one or more other envelopes or through an elastic strap to treat larger anatomical areas of the human body. However, the device of this document has the disadvantage of proposing a discontinuous attachment between the adjacent envelopes of the device, which implies that there is, around this attachment, an area of the patient that will not be treated by the device because it is not in contact with the device. On the other hand, only longitudinal areas can be treated with this device. As a result, this device is not totally reliable and is not compatible with all types of anatomical surfaces of the human body.

The prior art also includes therapeutic devices, as described for example in document WO2008094485, used to treat injury and discomfort by using hot or cold therapy applied to an individual or to an animal. In addition, this device uses a vibrating apparatus to therapeutically massage or stimulate the target area. Indeed, this document proposes a therapeutic device capable of providing temperature modulation and vibration. This therapeutic device includes both openings or that are adapted to receive removable hot or cold packs, and pouches to receive a vibrating apparatus. According to the embodiments of this document, these openings, pockets or pouches are either arranged only in a longitudinal plane; or arranged in a longitudinal plane and a plane perpendicular to said longitudinal plane; or this device includes a large gel pack completely covering a user's shoulder area. However, this device has the disadvantage of treating only a localized area of the body. To treat larger areas of the body, the dimensions of the device must be completely changed (this is the case with the device covering a user's shoulder). As a result, this device is not adjustable and is not compatible with all types and sizes of anatomical surfaces or areas of the human body.

The prior art includes therapeutic delivery systems, as for example described in document WO2009156907, including a disposable single-use device or garment for selectively holding at least one therapeutic article against the user's body. Indeed, this document offers practical and customizable clothing to hold therapeutic items in close contact with the body. However, the clothing (such as a glove or sock) of this document has the disadvantage of not allowing a painful joint or anatomical area to be immobilized or stabilized as would a splint. In addition, the therapeutic article is not removable or reusable.

The prior art includes outdoor clothing designed to provide heat and protection in a cold environment, as for example described in document US2011041229. A mesh pocket design is used on various garments to contain removable heating packs that require sufficient oxygen supply to activate the heating effect of said heating pack. The mesh pocket of D3 defines a single compartment of different shapes and sizes. According to one embodiment of this document, a sweater includes a plurality of pockets spaced apart and arranged along a longitudinal axis to heat the body and two oblique axes to heat the sleeves of the garment. These pockets are spaced apart and therefore do not extend along a longitudinal axis continuously. This configuration implies that on the spaced areas of the garment, there will be no treatment. In addition, the garment (such as a glove, a pair of pants or a sweater) of this document also has the disadvantage of not allowing a painful joint or anatomical area to be immobilized or stabilized as would a splint.

In this context, it is advantageous to propose a solution to overcome the disadvantages of the prior art by improving the therapeutic splint for the treatment of one or more therapeutic areas.

### GENERAL DESCRIPTION OF THE INVENTION

The purpose of the present invention is to overcome some of the disadvantages of the prior art by proposing a novel, reliable therapeutic splint that can be adapted for the treatment of all types of anatomical areas, particularly a patient's joint areas.

This goal is achieved by a therapeutic splint for the treatment of one or more anatomical areas of a patient, the splint including longitudinal housings extending along a longitudinal axis and arranged parallel to this longitudinal axis; one or more therapeutic cartridges suitable for being arranged in said housings, characterized in that
the splint further includes at least one transverse and/or oblique housing extending along one or more transverse and/or oblique axes, respectively, with respect to said longitudinal axis of the splint,
the transverse and/or oblique housing is arranged in a predetermined manner according to said anatomical area(s) of the patient, and
the arrangement of the cartridge is adjustable in said housings according to said anatomical area(s) to be treated.

According to one of the features, the one or more cartridges have a predetermined shape and size and are adapted to the dimensions of the longitudinal housings and the transverse and/or oblique housing of the splint.

According to one of the features, the cartridge includes a thermotherapy effect for thermal transfer treatment.

According to one of the features, at least one heating effect cartridge is arranged in said longitudinal housing and/or at least one cooling effect cartridge is arranged in said transverse and/or oblique housing of the splint.

According to one of the features, the cartridge has a straight or curved shape.

According to one of the features, the cartridge is reusable.

According to one of the features, the splint includes means for attachment with one or more other adjacent splints to treat a larger anatomical area.

According to one of the features, the attachment means include a fastener, a hook, a staple, laces, a zipper, Velcro tape (scratch), a self-adhesive fabric or combinations thereof.

According to one of the features, the attachment means extend over the entire circumference of the splint, parallel to the longitudinal axis.

According to one of the features, the splint includes one or more immobilization means, such as a metal plate, arranged in said housings, in order to totally or partially immobilize the anatomical area(s) of the patient.

According to one of the features, the splint is a therapeutic effect immobilization wrap and is wrapped around the anatomical area to be treated.

Other features, objects and advantages of the invention will become clearer after reading the description, drawings and claims.

### DESCRIPTION OF THE DRAWINGS

Other features and advantages of the present invention will become clearer when reading the following description, with reference to the accompanying drawings, wherein:

FIGS. 1a and 1b are schematic views of a therapeutic splint according to one embodiment of the invention, and a therapeutic splint attached to a second adjacent splint according to another embodiment of the invention;

FIG. 2 is a schematic view of a therapeutic splint attached to a second adjacent splint according to another embodiment of the invention with the anatomical members of a human body in the background;

FIG. 3 is a schematic view of a therapeutic splint attached to a second adjacent splint according to another embodiment of the invention, with the elbow, forearm and hand in the background;

FIG. 4 is a schematic view of a therapeutic splint according to another embodiment of the invention, to treat an inflammation of the synovium;

FIG. 5 is a schematic view of a therapeutic splint attached to two other adjacent splints according to another embodiment of the invention, to cover a larger inflamed joint area.

In the different figures, the same reference characters refer to identical or similar elements.

### DESCRIPTION OF THE PREFERRED EMBODIMENTS OF THE INVENTION

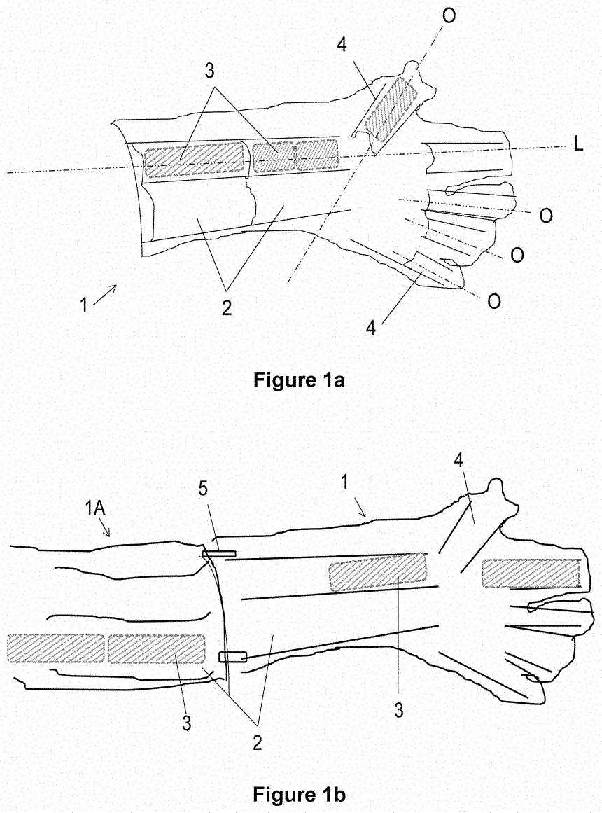

Various embodiments of the invention will now be described with reference to FIGS. 1 to 5.

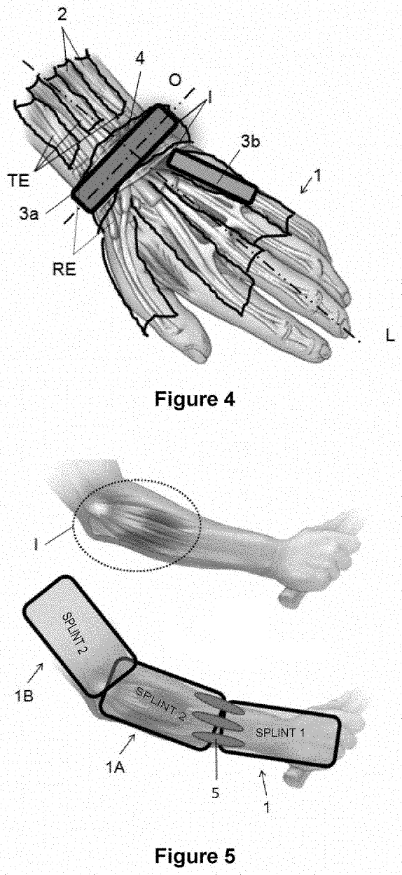

The present invention relates to a therapeutic splint (1) for the treatment of one or more anatomical areas of a patient. For example, these are any type of anatomical areas of the human body, such as, for example, a painful or unstable or healing anatomical joint or segment. Preferably, it is one or more joint areas of a patient. By “Joint or articulation area”, we mean an area or region of contact (or joint or connection) between two or more rigid elements (for example between two or more bone ends) that provides relative mobility of these elements or their interlocking in a fixed position. Among the different types of joints, the splint of the present invention can treat, for example, synovial joints that have high mobility, and can be held by ligaments delimiting a cavity (or joint capsule) containing a viscous and lubricating substance (called synovia). The bone ends in contact are covered with hyaline cartilage. Examples of this type of joint are present in the elbow (FIGS. 3 and 5), wrist (FIG. 4), knee, shoulder, etc. By “Therapeutic splint”, we refer to a medical device intended to immobilize and/or stabilize a joint or an anatomical area in order to treat said painful joint or area. Thus, a garment (such as a glove, a pair of pants, socks, a sweater, etc.) cannot be considered as a splint within the meaning of the present invention, since a garment is generally supple and flexible, and does not immobilize an anatomical area, preferably a joint area, and/or maintain the garment in close contact with a part of the human body or an anatomical area.

The splint (1) includes longitudinal housings (2), for example as shown in FIG. 1. The splint (1) extends along a longitudinal axis (L) and said housings (2) are arranged substantially parallel to this longitudinal axis (L). The splint (1) further includes at least one substantially transverse and/or oblique housing (4) which extends along one or more transverse and/or oblique axes (O), respectively, with respect to the longitudinal axis (L) of the splint (1). The transverse and/or oblique housing (4) is arranged in a predetermined manner according to the orientation of the various anatomical elements of the patient (for example tendons, muscles, bones, etc.), so as to facilitate and improve the healing of these anatomical elements. Indeed, the transverse and/or oblique housing(s) (4) can be arranged according to a straight and/or curved path or trajectory of a given anatomical area of the human body. Indeed, it is well known that anatomical limbs or structures are organized according to different reference planes and axes defined in relation to a standard position of the human body. Different anatomical structures within the same body part (for example arms, hands, legs, feet, etc.), can be arranged along the same axis or along several axes, which may or may not be parallel to each other, such as a longitudinal (or vertical) axis perpendicular to the ground when the person is standing, a transverse axis perpendicular to the longitudinal axis, a sagittal axis crossing the body from front to back and perpendicular to the longitudinal and transverse axes, and adding an oblique axis that is oblique at a variable angle (for example between 5-85°) to the longitudinal axis. Thus, these transverse and/or oblique housings are configured to, inter alia, specifically treat and/or stabilize painful joints consisting of complex structures (ligaments, tendons, membranes, cartilage, connective tissue, synovium, etc.), such as, for example and without limitation, hand osteoarthritis, wrist tendonitis, ankle sprain, etc. As shown for example in FIG. 3, an oblique housing can be arranged to specifically treat the muscle M4 (which may be the extensor carpi radialis longus, for example) between the arm and forearm. For example, the splint according to the configuration in FIG. 3, can treat both the muscle M4 arranged obliquely and the muscle M5 (which may be the biceps brachii muscle, for example) arranged longitudinally.

In some embodiments, the transverse and/or oblique housings of the splint are arranged so as to cover an area or region that extends around a joint to be treated. For example, and without limitation, the transverse and/or oblique housings of the splint may be oriented in a plane substantially perpendicular and/or oblique to a longitudinal plane of a joint, for example as mentioned in the present application.

These housings (2, 4) are configured to receive one or more therapeutic cartridges (3). One or more cartridges (3) may be inserted into one of said housings (2, 4) or simultaneously into several housings (2, 4), in order to obtain an arrangement adapted to the treatment of a specific anatomical area of the human body. The splint (1) can be arranged around a hand, as shown for example in FIG. 1a, and the cartridges (3) can be arranged in the upper longitudinal housings (2) and in one of the upper oblique housings (4), corresponding to the thumb of the hand (in this example), so as to specifically treat the upper areas (M1, M2) of the hand, starting from the thumb and then the wrist to the front arm. As shown for example in FIG. 4, the splint may include a housing (4) that is transverse in a substantially transverse axis (O) in relation to the longitudinal axis (L), for treating a joint area, particularly synovial inflammation (I). Indeed, the housings (2, 4) of the splint (1) in FIG. 4, suitable for receiving cartridges (3, 3a, 3b) are arranged so as to specifically treat all types of injuries of the junction between the tendons (or ligaments) and the muscles of the wrist. Thus, a cartridge (3a) can be arranged in the transverse housing (4) of the splint, for example according to the configuration in FIG. 4, to treat synovial inflammation (I) of the wrist that inflames the extensor retinaculum (RE) that holds the extensor tendons (TE) of the extensor muscles in place. One or more other cartridges (3b) can be arranged in the other housings (2, 4), in a way that is modular and adjustable by the patient, for example to treat inflamed or painful extensor tendons (TE). The longitudinal, transverse and/or oblique arrangement of the housings (2, 4) of the splint has the advantage of arranging the cartridges (3) in said housings (2, 4) in a modular and adjustable manner according to the use of the splint, in particular according to said anatomical area(s) to be treated. “Modular cartridge” means an insertion of one or more cartridges into the splint housings that can be modified, alternated, regulated or adjusted by a patient according to the painful areas to be relieved/treated or, in other words, a variable arrangement of cartridges in the housings of the splint. In some embodiments, as shown for example in FIGS. 1a, 1b, 2 and 3, the splint (1) has a therapeutic effect immobilization wrap or cuff that can be wrapped around the anatomical area to be treated. Immobilizing an anatomical area with the splint accelerates the treatment or healing of the immobilized anatomical area. This configuration of the splint in the form of a wrap or cuff also reduces its size, provides more comfort and facilitates its use for the patient. Various embodiments of the splint provide that it is also adapted to any other part of the human body, such as the shoulder, knee or foot. The example of the hand is therefore not restrictive and is used here to demonstrate the advantage of oblique and/or transversal housings.

In some embodiments, the cartridge (3) has a predetermined shape and dimension adapted to the dimensions of the longitudinal housings (2) and/or of the transverse and/or oblique housing (4) of the splint (1). This configuration allows the cartridge to be arranged in the housing in a fixed and stable manner and limits the movement of the cartridge out of said housing, for example when the patient makes sudden or large-amplitude movements that may dislodge the cartridge from the splint housing. Thus, the cartridge can have a predetermined size and shape and be specifically adapted to a localized anatomical area or a larger anatomical area depending on the treatment desired by the patient. In some embodiments, the cartridges (3) can be straight or curved depending on the anatomical area to be treated. A straight cartridge can be easily arranged in the housings that follow a straight trajectory of an anatomical area, while the curved cartridge can be easily arranged in the housings that follow a curved trajectory around an anatomical area of the human body, for example and not restrictively as shown in FIG. 3, and will therefore allow this anatomical area to be optimally treated. Curved cartridges can, for example, be used to specifically treat an area or limb of the elbow, knee or ankle. In some embodiments, the cartridge (3) is reusable. Indeed, a cartridge that has treated an anatomical area can be reused and readjusted to treat a second different anatomical area, so as to reduce the number of cartridges that can make the splint cumbersome.

In some embodiments, the cartridge (3, 3a, 3b) includes a thermotherapy effect for thermal transfer treatment. Indeed, for example, and without limitation, a cartridge or similar packaging includes a product to cool (called a “cold cartridge”) or to warm (called a “hot cartridge”) the painful anatomical areas of the patient. These cold and hot cartridges can speed up and improve the healing process or in some cases stimulate the repair process. For example, and without limitation, for a patient suffering from neuralgia or injury during sports activities, cold cartridges can be inserted into the splint housings to specifically relieve and treat the affected area.

In some embodiments, the splint (1) includes at least one heating effect cartridge (3b) which can be arranged in said longitudinal housing (2) and/or at least one cooling effect cartridge (3a) which can be arranged in said transverse and/or oblique housing (4) of the splint. As shown for example in FIG. 4, the use of a cooling or cold cartridge (3a) in the transverse housing (4) can stop the inflammation or the progression of the inflammation around or on the joint area to be treated (by the extensor retinaculum). In this configuration, a warming or hot cartridge (3b) can be synergistically combined to relieve or treat said joint area and/or the areas near the painful joint area.

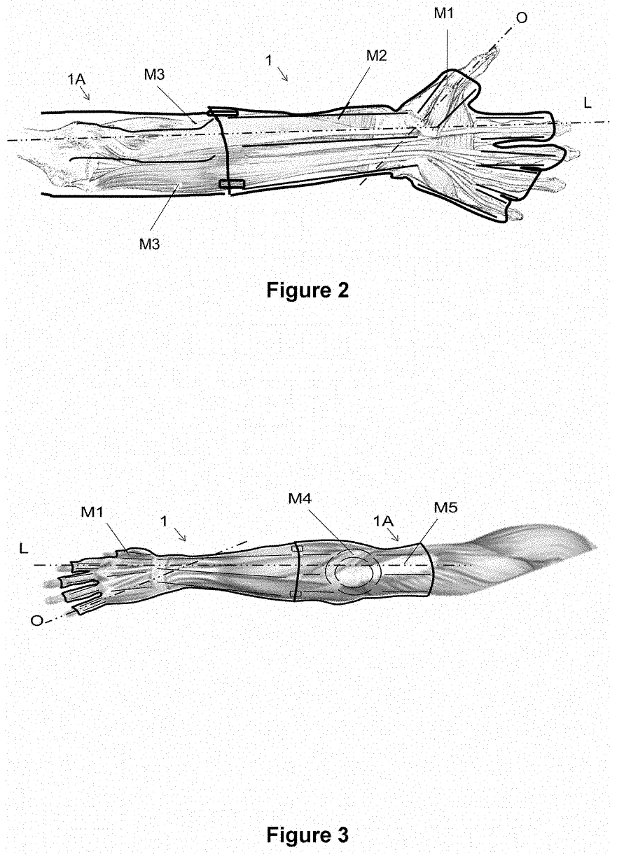

In some embodiments, the splint (1) includes means (5) for attachment with one or more adjacent splints (1, 1A) to treat a larger anatomical area. For example, as shown in FIG. 2, the splint (1) is attached with an adjacent splint (1A) to cover the hand, wrist and forearm, for example to simultaneously treat the upper anatomical areas (M1, M2, M3) of the forearm and hand. Similarly, for example as shown in FIG. 3, the splint (1) can also be attached with an adjacent splint (1A) so that the upper anatomical areas (M1, M4, M5) of the hand and elbow are treated simultaneously. As shown for example in FIG. 5, a splint (1) can be attached to two other adjacent splints (1A, 1B) to cover a larger painful joint area, such as an inflammation (I) in the elbow that may extend to the muscles of the patient's forearm. In some embodiments, the attachment means (5) include a fastener or a hook, a staple, laces, a zipper, Velcro tape (scratch), a self-adhesive fabric or any combination of at least two of these means. These attachment means allow the splints (1, 1A) to be quickly, easily and securely attached to each other. In some embodiments, the attachment means (5) extend over the entire circumference of the splint (1, 1A), parallel to the longitudinal axis (L), so as to allow a stable and reliable attachment between the splints (1, 1A).

In some embodiments, the splint (1) includes one or more means of immobilization of one or more predetermined anatomical areas or surfaces of the patient. Indeed, it is sometimes necessary to totally or partially immobilize certain parts of the human body, in order to avoid joint movements to promote healing. These immobilization means are arranged in the longitudinal housings (2) and/or the transverse and/or oblique housings (4) of the splint (1) according to the anatomical area(s) of the patient to be immobilized. This immobilization means includes a rigid part of adapted and variable shape and size to be inserted into said housings (2, 4) of the splint, for example, and without limitation, a metal plate or strip. The immobilization means can be arranged at a predetermined angle to immobilize, for example, the wrist for the treatment of carpal tunnel syndrome. Thus, the immobilization means can be arranged in said housings in a modular manner to adjust the maximum angle of movement (flexion/extension) of the limb of the human body and allow a movement of controlled amplitude. In certain embodiments, the splint (1) may include both a cartridge (3) in one or more of the splint housings (2, 4) and an immobilization means in one of the other housings (2, 4) of the splint, so as to immobilize one part of the body and treat the other part of the body with the therapeutic cartridge.

The combination of both longitudinal and transverse and/or oblique housings, with the modular cartridges of the therapeutic splint of the present invention, makes it possible to provide and carry out immobilization, compression and therapeutic treatment (for example cooling and/or heating) of one or more joint areas to be treated. The splint can be adapted, for example by attaching several adjacent splints, to treat and heal several positions of a patient's upper limb (for example, elbow, wrist and hand) and/or lower limb. Finally, the specific configuration of the splint of the present invention may also be used to treat joint diseases or injuries, such as carpal tunnel syndrome, tendonitis and/or rheumatism.

It should also be noted that the term “substantially” is regularly used in the present description, particularly in relation to a feature such as an orientation or a direction, so as to indicate that the feature concerned may in fact be slightly different and not exactly as designated (for example, the expression “substantially parallel” should be interpreted as “at least approximately parallel” because it may be possible to choose an orientation that is not exactly perpendicular so that it can nevertheless perform substantially the same function). In addition, terms such as the term “substantially” used in the present application may also be interpreted as defining that the technical feature may be “in general” (“generally”), and often “preferably”, as indicated, but that other embodiments or configurations may be within the scope of the present invention.

The present application describes various technical features and advantages with reference to the figures and/or to various embodiments. The skilled person will understand that the technical features of a given embodiment may in fact be combined with features of another embodiment unless the opposite is explicitly stated or it is obvious that these features are incompatible or that the combination does not provide a solution to at least one of the technical problems mentioned in the present application. In addition, the technical features described in a given embodiment may be isolated from the other features of that embodiment unless the opposite is explicitly stated.

It must be obvious to persons skilled in the art that the present invention allows embodiments in many other specific forms without departing from the scope of the invention as claimed. Consequently, the present embodiments must be considered by way of illustration, but may be modified within the scope defined by the scope of the accompanying claims, and the invention must not be limited to the details given above.

1. Therapeutic splint for the treatment of one or more joint areas of a patient, the splint including longitudinal housings extending along a longitudinal axis (L) and arranged parallel to this longitudinal axis (L); one or more modular cartridges with therapeutic effect suitable for being arranged in said housings according to said joint area(s) to be treated, wherein:
the splint further includes at least one transverse and/or oblique housing extending along one or more transverse and/or oblique axes (O), respectively, with respect to said longitudinal axis (L) of the splint,
the transverse and/or oblique housing is arranged so as to cover an area extending around a joint to be treated, and
the modular cartridge or cartridges are arranged in said transverse and/or oblique housings according to said joint area(s) to be treated.

2. Therapeutic splint according to claim 1, wherein the cartridge(s) have a predetermined shape and size and are adapted to the dimensions of the longitudinal housings and of the transverse and/or oblique housing of the splint.

3. Therapeutic splint according to claim 1, wherein the cartridge includes a thermotherapy effect for heat transfer treatment.

4. Therapeutic splint according to claim 3, wherein at least one heating effect cartridge is arranged in said longitudinal housing and/or at least one cooling effect cartridge is arranged in said transverse and/or oblique housing of the splint.

5. Therapeutic splint according to claim 1, wherein the cartridge has a straight or curved shape.

6. Therapeutic splint according to that claim 1, wherein the cartridge is reusable.

7. Therapeutic splint according to claim 1, wherein the splint includes an attachment with one or more other adjacent splints to treat a larger anatomical area.

8. Therapeutic splint according to claim 7, wherein the attachment is at least one of a fastener, a hook, a staple, laces, a zipper, Velcro tape (scratch), a self-adhesive fabric or combinations thereof.

9. Therapeutic splint according to claim 7, wherein the attachment extends over the entire circumference of the splint, parallel to the longitudinal axis.

10. Therapeutic splint according to claim 1, wherein the splint includes one or more immobilization means arranged in said housings, so as to totally or partially immobilize the anatomical area(s) of the patient.

11. Therapeutic splint according to claim 1, wherein the splint is a therapeutic effect immobilization wrap and is wrapped around the anatomical area to be treated.
